(19) United States
(12) Patent Application Publication
(10) Pub. No.: US 2022/0378766 A1
(43) Pub. Date: Dec. 1, 2022

(54) MODULATING EXPRESSION LEVEL OF A GENE ENCODING AN UNCOUPLING PROTEIN BY TREATING A HUMAN SUBJECT WITH A NITROXIDE

(71) Applicant: Louis HABASH, Irvine, CA (US)

(72) Inventor: Louis HABASH, Irvine, CA (US)

(21) Appl. No.: 17/330,155

(22) Filed: May 25, 2021

(51) Int. Cl. A61K 31/445

(52) U.S. Cl. CPC A61K 31/445

(57) ABSTRACT

Some embodiments disclosed herein include a method for increasing an expression level of a gene. The methods include identifying a human subject having a decreased expression level of UCP3; and administering to the human subject an effective amount of a nitroxide antioxidant, whereby expression level of the gene is increased.

### BACKGROUND

### Field

The present disclosure relates generally to the field of modulation of gene expression and more particularly to increasing expression levels of one or more genes relating to uncoupling protein 3 by treating human subjects with a nitroxide.

### Description of the Related Art

Diseases and conditions are treatable by adjusting the expression levels and activities of key genes in the body. Gene expression irregularities, whether overexpressed, activated, under expressed or inhibited underlie the development and progression of disease and condition. Some diseases are characterized by deficient expression of certain genes while other diseases result from over expression of certain genes. A disease resulting from irregular gene expression can be prevented, treated, or reversed by administering a nitroxide antioxidant to target and correct the expression levels of the genes.

Expression levels of genes are often naturally controlled in an appropriate way, but sometimes natural control of gene expression fails. For example, in cancer, genes providing instructions for cell growth are activated or switched on, when they should be off. Autoimmune diseases and aging are other examples of diseases and conditions that result from irregular gene expression. As cells age, the natural control of gene expression deteriorates promoting several diseases and conditions such as inflammation, chronic pain, infections, neurodegenerative disease, neurological disorders, skin diseases, etc. It is essential to identify the irregular expression of the genes involved in the cause of the disease and adjust the expression levels of those genes.

Often referred to as gene therapy, the targeting and correction of cellular dysfunction through adjusting the expression level of certain genes is necessary to prevent, treat, or reverse a disease or condition. Only by identifying key genes and developing therapeutics that altering the expression patterns of those genes can we prevent the development of the disease, reduce its effects once it has occurred, or reverse it all together.

One of the key genes involved in several diseases and conditions is uncoupling protein 3 (UCP3). When this gene is underexpressed it causes several diseases and conditions associated with the underexpression of the gene. Thus, correction of the underexpression of UCP3 genes is essential for treatment and prevention of the associated diseases and conditions.

### SUMMARY

Some embodiments disclosed herein provide methods for increasing or activating gene expression. The methods, in some embodiments, include identifying a human subject over the age of 35 and having a decreased expression level of UCP3; and administering to the human subject an effective amount of a nitroxide antioxidant resulting in a increased expression level of the gene. In some embodiments, the gene is UCP3. In some embodiments, the human subject is over the age of 20. In some embodiments, the human subject is over the age of 45. In some embodiments, the human subject is over the age of 55. In some embodiments, the human subject is over the age of 65. In some embodiments, the expression level of the gene in a skin tissue is increased by treatment. In some embodiments, the expression level of the gene in an adipose tissue is increased by treatment. In some embodiments, the expression level of the gene in blood is increased by treatment. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for increasing the expression level of a gene in a human subject in need thereof, comprising: identifying a human subject having a decreased expression level of UCP3; administering to the human subject an effective amount of a nitroxide antioxidant, whereby the expression level of UCP3 is increased. In some embodiments, the gene is UCP3. In some embodiments, the decreased expression level of the gene is age-related. In some embodiments, the human subject is over the age of 35. In some embodiments, the human subject is over the age of 45. In some embodiments, the human subject is over the age of 55. In some embodiments, the human subject is over the age of 65. In some embodiments, the decreased expression level of the gene is disease-related. In some embodiments, the disease is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the disease is age-related. In some embodiments, the expression level of the gene in a skin tissue is increased by treatment. In some embodiments, the expression level of the gene in an adipose tissue is increased by treatment. In some embodiments, the expression level of the gene in blood is increased by treatment with the nitroxide antioxidant. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for reducing risk of a disease in a human subject in need thereof, comprising: identifying a human subject over the age of 35 having an increased risk of a disease due to an decreased expression level of UCP3; administering to the human subject an effective amount of a nitroxide antioxidant, whereby the expression level of UCP3 is increased. In some embodiments, the disease is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is UCP3. In some embodiments, the human subject is over the age of 45. In some embodiments, the human subject is over the age of 55. In some embodiments, the human subject is over the age of 65. In some embodiments, the expression level of the gene in a skin tissue is increased by treatment. In some embodiments, the expression level of the gene in an adipose tissue is increased by treatment. In some embodiments, the expression level of the gene in blood is increased by treatment. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods comprising: identifying a human subject having or at risk of developing a cancer and in need of an increased expression level of a UCP3 gene; administering to the human subject an effective amount of a nitroxide antioxidant, whereby the expression level of the gene associated with uncoupling proteins and uncoupling protein activity is increased. In some embodiments, the cancer can be selected from the group consisting of bladder cancer, colorectal cancer, hepatocellular carcinoma, prostate carcinoma, and kidney carcinoma. In some embodiments, the gene is UCP3. In some embodiments, the cancer is age-related. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased by treatment. In some embodiments, the expression level of the gene in an adipose tissue is increased by treatment. In some embodiments, the expression level of the gene in blood is increased by treatment. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods comprising: identifying a human subject having or at risk of developing an autoimmune disease and in need of an increased expression level of a UCP3 gene; administering to the human subject an effective amount of a nitroxide antioxidant, wherein the expression level of the gene associated with uncoupling proteins and uncoupling protein activity is increased. In some embodiments, the autoimmune disease can be selected from the group consisting of rheumatoid/osteoid arthritis, systemic lupus erythematosus (SLE), inflammatory bowel disease, multiple sclerosis, atherosclerosis, and osteoporosis. In some embodiments, the gene is UCP3. In some embodiments, the autoimmune disease is age-related. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased by treatment. In some embodiments, the expression level of the gene in an adipose tissue is increased by treatment. In some embodiments, the expression level of the gene in blood is increased by treatment. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for a disease associated with a decreased expression level of uncoupling proteins and uncoupling protein activity is increased in a patient in need thereof, comprising: identifying a human subject having or at risk of developing a disease associated with a decreased expression of UCP3; administering to the human subject an effective amount of a nitroxide antioxidant, whereby the expression level of UCP3 is increased. In some embodiments, the disease can be selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is UCP3. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased by treatment. In some embodiments, the expression level of the gene in an adipose tissue is increased by treatment. In some embodiments, the expression level of the gene in blood is increased by treatment. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual in need thereof, comprising: identifying an individual over the age of 35 in need of an increased expression level of UCP3; and administering to the individual an effective amount of a nitroxide antioxidant to increase the level of expression of the gene associated with uncoupling proteins and uncoupling protein activity. In some embodiments, the gene is UCP3. In some embodiments, the human subject is over the age of 45. In some embodiments, the human subject is over the age of 55. In some embodiments, the human subject is over the age of 65. In some embodiments, the human subject has a decreased expression level of the gene. In some embodiments, the individual has or is at risk of developing an age-related condition. In some embodiments, the age-related condition comprises decreased senescence in a tissue. In some embodiments, the age-related condition comprises inhibition uncoupling proteins and uncoupling protein activity in a tissue. In some embodiments, the age-related condition comprises increased molecular heterogeneity. In some embodiments, the age-related condition comprises increased functional impairment in a tissue. In some embodiments, the expression level of the gene in a skin tissue is increased by treatment. In some embodiments, the expression level of the gene in an adipose tissue is increased by treatment. In some embodiments, the expression level of the gene in blood is increased by treatment. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual in need thereof, comprising: identifying an individual having a disease-related decreased expression level of UCP3; and administering to the individual an effective amount of a nitroxide antioxidant to increase the level of expression of the gene associated with uncoupling proteins and uncoupling protein activity. In some embodiments, the disease can be selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is UCP3. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased by treatment. In some embodiments, the expression level of the gene in an adipose tissue is increased by treatment. In some embodiments, the expression level of the gene in blood is increased by treatment. In some embodiments, the expression level of the gene in a neuronal tissue is increased by treatment. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual having or at risk of developing a condition due to aging, comprising: identifying an individual over the age of 35; and administering to the individual an effective amount of a nitroxide antioxidant, whereby the expression level of the gene associated with uncoupling proteins and uncoupling protein activity is increased. In some embodiments, the individual has an decreased expression level of the gene. In some embodiments, the gene is UCP3. In some embodiments, the condition is an age-related condition. In some embodiments, the age-related condition comprises increased senescence in a tissue. In some embodiments, the age-related condition comprises underactivation of UCP3 in a tissue. In some embodiments, the age-related condition comprises increased molecular heterogeneity. In some embodiments, the age-related condition comprises increased functional impairment in a tissue. In some embodiments, the age-related condition is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the human subject is over the age of 35. In some embodiments, the human subject is over the age of 45. In some embodiments, the human subject is over the age of 55. In some embodiments, the human subject is over the age of 65.

Some embodiments disclosed herein provide methods for increasing the expression level of a gene in a human subject in need thereof, comprising: identifying a human subject having a decreased expression level of UCP3; and delivering to the human subject an effective amount of a nitroxide antioxidant to increase the level of expression of the gene associated with uncoupling proteins and uncoupling protein activity. In some embodiments, the nitroxide antioxidant is 4-hydroxy-2,2,6,6-tetramethylpiperidine-1-oxyl. In some embodiments, the decreased expression level of the gene is age-related. In some embodiments, wherein the decreased expression level of the gene is cancer-related. In some embodiments, the decreased expression level of the gene is disease-related. In some embodiments, the decreased expression level of the gene is neurodegeneration-related. In some embodiments, the decreased expression level of the gene is infection related. In some embodiments, the increased the level of expression of the gene improves uncoupling activity and mitochondrial function. In some embodiments, the expression level of the gene is increased in a tissue selected from the group consisting of a skin tissue, an immune tissue, an adipose tissue, a pancreatic tissue, cardiac tissue, and a neuronal tissue by treatment.

Some embodiments disclosed herein provide methods for increasing an expression level, in an eukaryotic cell, of one or more genes encoding uncoupling proteins involved in mitochondrial uncoupling by contacting the eukaryotic cell with a nitroxide antioxidant. In some embodiments, the one or more genes is UCP3. In some embodiments, the nitroxide antioxidant is 4-hydroxy-2,2,6,6-tetramethylpiperidine-1-oxyl. In some embodiments, the eukaryotic cell is a cancer cell. In some embodiments, the expression level of the one or more genes is decreased in said cell in a tissue selected from the group consisting of a skin tissue, an immune tissue, an adipose tissue, a pancreatic tissue, cardiac tissue, and a neuronal tissue. In some embodiments, prior to said contacting, the eukaryotic cell exhibits an age-related decreased expression level of said one or more genes. In some embodiments, prior to said contacting, the eukaryotic cell exhibits a disease-related decreased expression level of said one or more genes. In some embodiments, prior to said contacting, the eukaryotic cell exhibits a neurodegeneration-related expression level of said one or more genes.

Some embodiments disclosed herein provide methods for improving chemotherapeutic response in a human subject comprising: contacting cancer cells in the subject with an effective amount of a nitroxide antioxidant whereby a level of expression of uncoupling proteins and uncoupling protein activity is increased in said cancer cells. In some embodiments, said cancer cells are known to have decreased UCP3 function. In some embodiments, the decreased expression level of one or more genes following treatment initiates apoptosis within one or more of said cancer cells. In some embodiments, the decreased expression level reduces or prevents resistance to other chemotherapeutic agents. In some embodiments, the nitroxide antioxidant is 4-hydroxy-2,2,6,6-tetramethylpiperidine-1-oxyl. In some embodiments, the gene is selected from the group consisting of UCP3.

Some embodiments disclosed herein provide methods for increasing uncoupling proteins and uncoupling protein activity in a human subject comprising: identifying a human subject known to have decreased UCP3 function; and delivering to the subject an effective amount of a nitroxide antioxidant, whereby a level of uncoupling proteins and uncoupling protein activity is increased. In some embodiments, the nitroxide antioxidant is 4-hydroxy-2,2,6,6-tetramethylpiperidine-1-oxyl. In some embodiments, decreased UCP3 function is age-related. In some embodiments, the decreased UCP3 function is cancer-related. In some embodiments, the decreased UCP3 function is disease-related. In some embodiments, the decreased UCP3 function is neurodegeneration-related. In some embodiments, the decreased UCP3 function is infection-related. In some embodiments, the decreased level of expression of the gene improves remodeling of damaged tissues. In some embodiments, the expression level of the gene is decreased in a tissue selected from the group consisting of a skin tissue, an immune tissue, an adipose tissue, a pancreatic tissue, cardiac tissue, and a neuronal tissue following treatment.

Some embodiments disclosed herein provide methods for treating a human subject having cancer comprising: delivering an effective amount of a nitroxide antioxidant to a human subject, wherein the human subject has previously been administered at least one chemotherapeutic agent, whereby a level of expression of uncoupling proteins and uncoupling protein activity is increased. In some embodiments, the human subject having cancer is identified with a decreased expression of UCP3. In some embodiments, the methods further comprise administering a promotor of a UCP3 to the human subject.

### DETAILED DESCRIPTION

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

All patents, applications, published applications and other publications referred to herein are incorporated by reference for the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

As used herein, the term “expression” means the detection of a gene product that is expressed or produced by a nucleic acid molecule by standard molecular biology methods, which gene product refers to e.g. an unspliced RNA, an mRNA, a splice variant mRNA, a polypeptide, a post-translationally modified polypeptide, a splice variant polypeptide etc., and specifically products made using an RNA gene product as a template, e.g. cDNA of the RNA.

As used herein, “differential expression” of a gene means that the expression of the gene is at a higher level (“increased expression”) or lower level (“decreased expression”) in a human subject suffering from a disease, for example cancers and autoimmune diseases. Differential expression includes both quantitative, as well as qualitative, differences in the temporal or cellular expression pattern in a gene or its expression products among, for example, normal and diseased cells, or among cells which have undergone different disease events or disease stages.

As used herein, “increasing the expression level” of a gene means causing the expression of the gene to decrease by treating the human subject with a compound, for example a nitroxide antioxidant, such that the expression level of the gene after treatment is lower than the expression level of the gene before treatment in the human subject.

As used herein, “delivering” a compound shall mean bringing that compound into contact with a relevant cell, tissue, or organism. Similarly, “contacting” shall mean that the compound contacts a relevant target, such as a tissue or cell or tumor. In either case, delivery or contact in an organism can be affected by directly administering the compound to the organism, or by administering a different compound to the organism, such as a prodrug that is converted in vivo to the desired compound. In short, these terms cover any action that leads to contact between the desired compound and a target cell, tissue, or organism.

The present disclosure describes methods of modulating gene expression in human subjects. However, this is illustrative only and not intended to be limiting. For example, the methods disclosed herein can be used for modulating gene expression in other vertebrates, such as but not limited to mammals, birds, reptiles, fish, and the like (with modifications wherein appropriate). Mammals and birds include most agricultural animals. Treatment of companion animals, e.g., dogs, cats, or birds is also contemplated.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### Human Subject Identification

The present disclosure relates to methods of treating alterations in gene expression, such as age-related, cancer-related, disease-related, neurodegeneration-related, and infection-related alteration in gene expression. Gene expression changes also play important roles in aging and serve as biomarkers of physiological decline and disease conditions, such as neurodegenerative diseases, and cancers. Therefore, one aspect of the present disclosure is methods of treating a human subject having an age-related, cancer-related, disease-related, neurodegeneration related, and/or infection-related decrease in gene expression levels, such as those genes associated with uncoupling proteins and uncoupling protein activity. In some embodiments, the human subject can be identified based on the human subject's age, gene expression level, family history, health conditions, medical history, habits, or a combination thereof.

Regardless of the cause of the upregulation, some common terminology can be used. In some embodiments, the expression level of a gene (e.g., UCP3) in a human subject is considered to be upregulated or increased if the increase in the expression level of that gene is statistically significant compared to that of a control or a reference. In some embodiments, the expression level of a gene (e.g., UCP3) in a human subject is considered to be upregulated or increased if the increase in the expression level of that gene is statistically significant compared to that of a control or a reference. The control or reference can be, for example, a normal healthy population, a population at large, a collection of individuals of the same age or condition or sex, or the same human subject at a different time (e.g., at an earlier time of life when the human subject does or does not have the disease or condition that results in the upregulation).

In some embodiments, a normal healthy population or a population at large can be a population having the same or similar gender, age, and/or race, compared to the human subject. In some embodiments, the expression level of the gene in the control or reference can be the mean or median expression level of the gene in control subjects in the control or reference subjects in the reference. The increase in expression level can be statistically significant if the probability of the observed difference occurring not by chance, the confidence level, is greater than a threshold. The threshold can be, or be about, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values.

In some embodiments, the increase in expression level can be, or be about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, or a number or a range between any two of these values. In some embodiments, the increase in expression level can be at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, or more.

In some embodiments, the human subject may have an age that is, is about, or is over 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old.

In some embodiments, the human subject is identified based on the human subject's expression profiles of UCP3. Non-limiting exemplary methods for determining the human subject's expression profiles include: amplification techniques such as PCR and RT-PCR (including quantitative variants), hybridization techniques such as in situ hybridization, microarrays, blots, and others, and high throughput sequencing techniques like Next Generation Sequencing (Illumina, Roche Sequencer, Life Technologies SOLID™), Single Molecule Real Time Sequencing (Pacific Biosciences), True Single Molecule Sequencing (Helicos), or sequencing methods using no light emitting technologies but other physical methods to detect the sequencing reaction or the sequencing product, like Ion Torrent (Life Technologies). Non-limiting exemplary methods for determining the human subject's expression profiles include: binding techniques such as ELISA, immunohistochemistry, microarray and functional techniques such as enzymatic assays.

### Targeted Gene Expression Adjustment

Mitochondria are specialized organelles within the cell. Often referred to as the “powerhouse” of the cell, the mitochondria is the location where cellular respiration occurs resulting in the productions of adenosine triphosphate (ATP). Major components of this energy production process are uncoupling proteins which serve to direct and pass charged particles throughout the cellular respiration process. In addition, mitochondria activity is vital for every aspect of cellular function. When a functional component of the mitochondria is inhibited or underexpressed, a cascade of cellular dysfunction causes the cell, tissue, and organism to be in a disease state.

All living organisms are comprised of cells that function individually as well as in combination with other cells to form larger and more complex structures such as tissue and organs. The operation of each cell is based on the genetic instructions provided by the DNA contained therein. DNA is arranged in a particular sequence referred to as a gene which is transcribed and translated into a functional product required for the operation of the cell.

Genes are expressed in a particular quantity based on the instruction provided by the DNA. In particular, gene expression describes transcription of gene encoding DNA sequences into complementary DNA (cDNA) and translation of cDNA into the functional products, such as proteins. Many factors, both internal and external, are involved in regulation of gene expression in cells. Such regulation can manifest in an adjustment of gene expression to increase or decrease a number of proteins made.

A condition or disease is identifiable based on such dysfunctional expression of genes within the cell. Whether the dysfunctional expression of the genes is due external influence on the cell or genetic aberrations, treating the dysfunctional expression is necessary to address the underlying cause of the condition or disease. Overexpression or under expression of a gene or genes often results in dysfunction of downstream actions controlled by the same. Whether the gene is a regulator of cellular function or a vital in a responsive mechanism, modulation of the gene expression is a fundamental directive in addressing the foundational issues associated with many diseases and conditions. Treatments for a disease or condition may be directed at addressing a manifestation or symptom of the disease. However, the underlying disease will be permitted to remain resulting in subsequent presentation of the previously treated symptoms. Therefore, it is essential to correct or reinforce the underlying cause of the disease. Ultimately, the treatment of the disease or condition requires targeting and modulating the expression level of the gene or genes that are overexpressed or under expressed.

In a healthy state, a predictable amount of ATP is produced and the uncoupling proteins regulate flow of protons out of the mitochondria for thermal regulation of the cell. When these proteins are underexpressed or inhibited, their corresponding function is reduced, which results in disease progression. Whether the inhibition of these proteins is reactive, or due to a genetic abnormality, it is the dysfunctional expression that defines a disease state for the subject.

Certain conditions, such as cardiovascular diseases, diabetes, obesity, and aging are associated with (e.g., causes or caused by) underexpression of uncoupling protein encoding genes resulting in the inhibition, inactivity, and disfunction of vital cellular processes within cells and tissues. Thus, modulation of underexpressed uncoupling protein genes is essential for treatment and prevention of certain conditions.

### Genes Associated with Uncoupling Proteins and Uncoupling Protein Activity

In some embodiments, administering to the human subject an effective amount of the nitroxide antioxidant results in an increased expression level of a gene, for example UCP3. Therefore, some embodiments disclosed herein provide methods for treating an individual in need thereof, comprising identifying an individual having a disease-related decreased expression level of UCP3; and administering to the individual an effective amount of a nitroxide antioxidant to increase the level of expression of UCP3. Some embodiments disclosed herein provide methods for treating an individual in need thereof, comprising identifying an individual in need of an increased expression level of a UCP3 gene; and administering to the individual an effective amount of a nitroxide antioxidant to increase the level of expression of UCP3. Some embodiments disclosed herein provide methods for treating an individual in need thereof, comprising: administering to the individual, known to have a disease-related decreased expression level of UCP3, an effective amount of a nitroxide antioxidant to increase the level of expression of UCP3. Some embodiments disclosed herein provide methods for treating an individual in need thereof, comprising: administering to an individual, known to be in need of an increased expression level of a UCP3 gene, an effective amount of a nitroxide antioxidant to increase the level of expression of uncoupling proteins and uncoupling protein activity. In addition to increasing the expression of UCP3, administration of the nitroxide antioxidant may result in an increase to uncoupling protein 1 (UCP1). UCP1 and UCP3 are members of the uncoupling protein subfamily and are localized in the inner mitochondrial membrane. Studies have shown that expression of UCP3 directly correlates to UCP1 abundance in brown adipose tissue. ((Karolina E. Hilse, Anastasia V. Kalinovich, Anne Rupprecht, Alina Smorodchenko, Ute Zeitz, Katrin Staniek, Reinhold G. Erben, Elena E. Pohl. The expression of UCP3 directly correlates to UCP1 abundance in brown adipose tissue. Biochimica et Biophysica Acta (BBA)—Bioenergetics, Volume 1857, Issue 1, 2016, Pages 72-78.)

Non-limiting examples of diseases associated with altered level of uncoupling proteins and uncoupling protein activity include cancer; breast cancer; lung cancer; kidney cancer; cancers of the ovary and uterus; cancer of the central nervous system; cancers of the head and neck; melanoma; lymphomas; leukemia; neurological disorders; Alzheimer's disease; Parkinson's disease; Huntington's disease; amyotrophic lateral sclerosis; stroke; cardiovascular disorders; ischemia; heart failure; infections, infectious diseases; bacterial infections; inflammatory responses; viral infections; autoimmune diseases; systemic lupus erythematosus; autoimmune lymphoproliferative syndrome; rheumatoid arthritis; and thyroiditis.

The gene associated with uncoupling protein 3 can be UCP3, UCP1, UCP2, UCP4, UCP5, or a homologue thereof. For example, the treatment results in increased expression levels of UCP3. The increased expression level of UCP3, increases uncoupling proteins and uncoupling protein. The increased level of UCP3 results in a decrease in or disappearance of signs and symptoms of a disease associated with decreased UCP3 function, including the curing of the disease associated with decreased UCP3 function. In some embodiments, the increased expression level of UCP3, increases the level of uncoupling proteins and uncoupling protein activity. The increased level of uncoupling proteins and uncoupling protein activity results in a decrease in or disappearance of signs and symptoms of the disease associated with decreased UCP3 function, including the curing of the disease associated with decreased UCP3 function. In some embodiments, the increased level of uncoupling proteins and uncoupling protein activity inhibits, suppress, prevent, or reverse the disease or the symptoms associated with the disease.

### Mitochondria and Uncoupling Proteins

Mitochondria are the cellular organelles where respiration occurs. They contain two compartments bounded by inner and outer membranes. The outer membrane is permeable to small metabolites, whereas the permeability of the inner membrane is controlled to maintain the high electrochemical gradient created by the mitochondrial respiratory chain that is necessary for energy conservation and ATP synthesis in mitochondria. The inner membrane transports anion substrates such as ADP, ATP, phosphate, oxoglutarate, citrate, glutamate, and malate. The reactions of the citric acid cycle, fatty acid oxidation, and several steps of urea synthesis and gluconeogenesis also take place in mitochondria. Energy produced by mitochondrial respiration is used for ATP synthesis by a complex mechanism referred to as “oxidative phosphorylation.” In addition to oxidative phosphorylation and metabolic pathways, mitochondria are involved in thermogenesis, radical production, calcium homeostasis, protein synthesis, and apoptosis. Although respiration is coupled with ADP phosphorylation, this coupling is less than perfect and may be partially or very partially loose. The uncoupling proteins (UCPs) are particular mitochondrial transporters of the inner membrane that appear to be controlling the level of respiration coupling. Several reviews devoted to UCPs have been published in the last few years.

Uncoupling proteins are mitochondrial transporters present in the inner membrane of mitochondria. They are found in all mammals and in plants. They belong to the family of anion mitochondrial carriers including adenine nucleotide transporters. The term “uncoupling protein” was originally used for UCP1, which is uniquely present in mitochondria of brown adipocytes, the thermogenic cells that maintain body temperature in small rodents. In these cells, UCP1 acts as a proton carrier activated by free fatty acids and creates a shunt between complexes of the respiratory chain and ATP synthase. Activation of UCP1 enhances respiration, and the uncoupling process results in a futile cycle and dissipation of oxidation energy as heat. UCP2 is ubiquitous and highly expressed in the lymphoid system, macrophages, and pancreatic islets. UCP3 is mainly expressed in skeletal muscles. In comparison to the established uncoupling and thermogenic activities of UCP1, UCP2 and UCP3 appear to be involved in the limitation of free radical levels in cells rather than in physiological uncoupling and thermogenesis. Moreover, UCP2 is a regulator of insulin secretion and UCP3 is involved in fatty acid metabolism. (The Biology of Mitochondrial Uncoupling Proteins, Sophie Rousset, Marie-Clotilde Alves-Guerra, Julien Mozo, Bruno Miroux, Anne-Marie Cassard-Doulcier, Frederic Bouillaud, Daniel Ricquier, Diabetes Feb 2004, 53 (suppl 1) S130-S135; DOI: 10.2337/diabetes.53.2007.S130

### UCP3

Mitochondrial uncoupling protein 3 (UCP3) is encoded by the UCP3 gene and is a member of the larger family of mitochondrial anion carrier proteins (MACP). Uncoupling proteins facilitate the transfer of anions from the inner to the outer mitochondrial membrane and transfer of protons from the outer to the inner mitochondrial membrane, reducing the mitochondrial membrane potential in mammalian cells. Uncoupling proteins dissipate the proton gradient, releasing stored energy as heat. These proteins also participate in energy expenditure, thermogenesis, regulation of free fatty acids, and reduction of reactive oxygen species.

Although UCP3 is expressed predominantly in skeletal muscle, it is also found in brown adipose tissue, cardiac muscle, and in certain areas of the brain (Boss, Olivier, Samec, Sonia, Paoloni-Giacobino, Ariane, Rossier, Colette, Dulloo, Abdul, Seydoux, Josiane, Muzzin, Patrick and Giacobino, Jean-Paul (1997), Uncoupling protein-3: a new member of the mitochondrial carrier family with tissue-specific expression, FEBS Letters, 408, doi: 10.1016/S0014-5793(97)00384-0). UCP3 is involved in, among other functions, energy metabolism regulation and weight control. For example, studies have shown that UCP3 plays an important part in modulating the use of lipid and glucose as energy substrate. Gaining better understanding of the role UCPs play in controlling and maintaining energy substrate oxidation could help to manage obesity.” (Oliveira B A P, Pinhel M A S, Nicoletti C F, Oliveira C C, Quinhoneiro D C G, et al. (2016) UCP1 and UCP3 Expression Is Associated with Lipid and Carbohydrate Oxidation and Body Composition. PLOS ONE 11(3):,e0150811. https://doi.org/10.1371/journal.pone.0150811)

### Heart Disease

UCP3 has been shown to have antioxidant properties known to stimulate long-chain fatty acid (LCFA) metabolism in muscle cells, for example is cardiac muscle cells. Myocardial UCP3 deficiency is a mechanism for the poor prognosis of type 2 diabetic patients following myocardial infarction. In prediabetic subjects and type 2 diabetic patients, skeletal muscle UCP3 content is decreased by almost 50%.

In the United States alone, more than 35 million individuals are suffering from diabetes mellitus. By 2030, this diabetes epidemic is expected to extend to more than 54 million Americans and will lead to nearly 400,000 deaths annually. Cardiovascular disease is the most prevalent cause of mortality in diabetic patients. Type 2 diabetes mellitus represents >95% of all diabetes cases. The hyperinsulinemia and reduced insulin sensitivity which characterize this metabolic disorder are linked to a two- to fourfold increase in the risk for ischemic heart disease. (Edwards, K. S., Ashraf, S., Lomax, T. M. et al. Uncoupling protein 3 deficiency impairs myocardial fatty acid oxidation and contractile recovery following ischemia/reperfusion. Basic Res Cardiol 113, 47 (2018). https://doi.org/10.1007/s00395-018-0707-9)

Reactive oxygen species (ROS) has also been found to be significantly increased subjects with heart disease. ROS production was increased in UCP3 knockout. Mitochondrial ROS production has previously been reported to increase in hearts of ucp3−/−mice and to mediate cardiac injury following MI. This increase in mitochondrial ROS generation was not accompanied by a compensatory increase of cellular antioxidant defenses in UCP3-deficient hearts as the activity of CAT, SOD, and GPx remained unchanged when compared to cardiac tissue from ucp3+/+rats. Id.

After a myocardial infarction (MI), damage to cardiac tissue was observed in healthy tissue, and UCP3 deficient tissue. However, compared with wild type, UCP knockout mice displayed a significant worsening in cardiac function after coronary artery ligation and a more pronounced increase in the left ventricle weight-to-body weight ratio.

Furthermore, studies show increased apoptotic cell death in ucp3−/−hearts after coronary artery ligation UCP3 genetic deletion significantly increased the rate of apoptotic cell death 8 weeks after MI. Consistent with these results, cleaved caspase 3 and p53 levels were significantly higher in ucp3−/−hearts after MI, whereas AKT phosphorylation was significantly reduced. Taken together, these data suggest that ucp3 levels regulate cell survival and infarct size after acute MI by modulating mitochondrial structural and functional adaptations to myocardial ischemia.

Mitochondrial ucp3 plays a pivotal role in the regulation of mitochondrial function, ROS production, and cell survival in the ischemic heart, modulating infarct size, cardiac remodeling, and survival after MI. These beneficial effects could be related to the ability of ucp3 to promote mitochondrial uncoupling and control ROS production and represent novel therapeutic targets in patients with coronary artery disease.” (Cinzia Perrino, M D, PhD; Gabriele G. Schiattarella, M D; Anna Sannino, M D, et. al. Genetic Deletion of Uncoupling Protein 3 Exaggerates Apoptotic Cell Death in the Ischemic Heart Leading to Heart Failure)

UCP3 is found at even higher levels in cardiac muscle, making heart an important organ for UCP3 function analysis. Several studies have shown that a decrease or loss of UCP3 in rodent knockout models diminished contractile heart function and increased ROS production and tissue damage following ischemia, while its upregulation during ischemia improved the recovery of cardiomyocytes and increased cardiac efficiency. (Mitochondrial uncoupling protein 2 (UCP2), but not UCP3, is sensitive to oxygen concentration in cells, Karolina E. Hilse, Anne Rupprecht, Kristopher Ford, Olena Andrukhova, Reinhold Erben, Elena E. Pohl, bioRxiv 2020.07.01.181768; doi: https://doi.org/10.1101/2020.07.01.181768).

In addition to the direct role of UCP3, studies have shown that UCP3 may play a central role in cardioprotective effect of Peroxisome proliferator-activated receptor a (PPARa) activation. PPARa), a member of the nuclear hormone receptor superfamily, is a transcription factor that regulates the expression of genes involved in cellular lipid metabolism. Myocardial PPARa-overexpression increased fatty acid uptake and oxidation with a concomitant decrease in glucose uptake and oxidation, and these metabolic changes were associated with left ventricular hypertrophy and systolic dysfunction. In contrast, pharmacologic activation of PPARa has been shown to confer myocardial protection against acute ischemia-reperfusion injury and acute activation of PPARa by pharmacologic agonists demonstrated cardioprotection against ischemia-reperfusion injury

### Diabetes and Insulin Resistance

UCP3 functioning to facilitate fatty acid oxidation and minimize ROS production. As impaired fatty acid metabolism and ROS handling are important precursors in muscular insulin resistance, UCP3 is an important therapeutic target in type 2 diabetes.

Insulin resistance in muscle is a primary component of type 2 diabetes, a disease with a prevalence that is increasing at an alarming rate in modern society. The importance of mitochondrial oxidative phosphorylation in the development of type 2 diabetes has recently been demonstrated through gene expression profiling studies in muscle of pre-diabetic, diabetic, and nondiabetic populations. Individuals with type 2 diabetes were shown to have reduced expression of genes for key proteins in oxidative metabolism and mitochondrial function in muscle. One of the mitochondrial proteins having decreased mRNA expression in pre-diabetic and diabetic subjects is uncoupling protein (UCP)3. Not only are the levels of mRNA for UCP3 decreased but also are the levels of UCP3 protein. (Physiological Increases in Uncoupling Protein 3 Augment Fatty Acid Oxidation and Decrease Reactive Oxygen Species Production Without Uncoupling Respiration in Muscle Cells, J. Darcy MacLellan, Martin F. Gerrits, Adrienne Gowing, Peter J. S. Smith, Michael B. Wheeler, Mary-Ellen Harper, Diabetes August 2005, 54 (8) 2343-2350; DOI: 10.2337/diabetes.54.8.2343).

Other studies have shown decreased levels of UCP3 are found in subjects with diabetes, in particular, type 2 diabetes. Type 2 diabetes is associated with impairments of glucose homeostasis. Increased expression of UCP3 was shown to improve and properly regulate glucose metabolism and homeostasis. Studies have shown that patients with type 2 diabetes have decreased levels of UCP3 protein content. (Uncoupling Protein 3 Content Is Decreased in Skeletal Muscle of Patients With Type 2 Diabetes, Patrick Schrauwen, Matthijs K. C. Hesselink, Ellen E. Blaak, Lars B. Borghouts, Gert, Schaart, Wim H. M. Saris, Hans A. Keizer, Diabetes Dec 2001, 50 (12) 2870-2873; DOI: 10.2337/diabetes.50.12.2870).

### Obesity

Obesity is a complex disease, with interactions between genetic predisposition, regulation of food intake and energy expenditure, food-seeking behavior, and the environment. Obesity has a strong association with type 2 diabetes mellitus (T2DM). The control of food intake, that is the size and frequency of meals, is a major factor in the determination of the individual's weight. Gastrointestinal functions such as gastric emptying and capacity (volume) also influence food intake and influence body weight. (Acosta, Andres et al. “Association of UCP-3 rs1626521 with obesity and stomach functions in humans.” Obesity (Silver Spring, Md.) vol. 23,4 (2015): 898-906. doi:10.1002/oby.21039)

As mentioned above, UCP3 plays an active role in non-shivering thermogenesis. The oxidation of fatty acids and pyruvate takes place in mitochondria, where energy is converted into ATP for use in cellular processes. Reducing equivalents are extracted from substrates and sequentially passed from electron donors (reductants) to acceptors (oxidants) along the mitochondrial respiratory chain to molecular oxy- gen. The electron transport system is located on the inner mitochondrial membrane, where oxidation steps are coupled by the transport chain to the extrusion of protons out of the matrix. This establishes an electro- chemical potential difference across the inner membrane and a motive force for proton reentry through F1F0-ATP synthase. ATP synthase captures the potential energy released upon proton reentry by converting ADP to ATP. In this manner, electron transport is coupled to oxidative phosphorylation. In a perfectly coupled system, protons only enter the mitochondrial matrix through ATP synthase in the presence of ADP; this form of respiration is classified as state 3 (i.e., O2 is consumed only in the presence of substrate and ADP). However, mitochondria can also be observed to use oxygen even in the absence of ADP, which occurs when protons leak back into the matrix. (The role of uncoupling protein 3 in human physiology. (W. Timothy Garvey Published Feb. 15, 2003, J Clin Invest. 2003; 111(4):438-441. https://doi.org/10.1172/JCI17835).

Understanding the metabolic factors that contribute to energy metabolism (EM) is critical for the development of new treatments for obesity and related diseases. Mitochondrial oxidative phosphorylation is not perfectly coupled to ATP synthesis, and the process of proton-leak plays a crucial role. Proton-leak accounts for a significant part of the resting metabolic rate (RMR) and therefore enhancement of this process represents a potential target for obesity treatment.((Busiello, Rosa A et al. “Mitochondrial uncoupling proteins and energy metabolism.” Frontiers in physiology vol. 6 36. 10 Feb. 2015, doi:10.3389/fphys.2015.00036).

UCP3 plays key regulatory roles in mitochondrial fatty acid oxidation and in preventing mitochondrial ROS-induced oxidative damage (Busiello, Rosa A et al. “Mitochondrial uncoupling proteins and energy metabolism.” Frontiers in physiology vol. 6 36. 10 Feb. 2015, doi:10.3389/fphys.2015.00036). Recent studies suggest that the primary role of UCP3 is in fatty acid metabolism and possibly in protecting the mitochondria against lipid-induced damage by reducing mitochondrial production of ROS and increasing the capacity to store energy as fat. Activation of UCP3 is indirectly regulated by norepinephrine and is dependent upon the availability of free fatty acids [FFAs]. Impaired mitochondrial function in adipocytes are linked directly to the development of metabolic diseases such as diabetes and insulin resistance, and mitochondrial genetic variants are associated with BMI in adults. (Acosta, Andres et al. “Association of UCP3 rs1626521 with obesity and stomach functions in humans.” Obesity (Silver Spring, Md.) vol. 23,4 (2015): 898-906. doi:10.1002/oby.21039).

### Chronic Obstructive Pulmonary Disease (COPD)

COPD, is a group of progressive lung diseases. The most common are emphysema and chronic bronchitis. Many people with COPD have both of these conditions. Emphysema, for example, slowly destroys air sacs in your lungs, which interferes with outward air flow. Bronchitis causes inflammation and narrowing of the bronchial tubes, which allows mucus to build up.

Studies have shown that UCP3 content is decreased in peripheral skeletal muscle of patients with chronic obstructive pulmonary disease and is related to disease severity. In particular, studies have shown significantly lower UCP3 content in peripheral skeletal muscle of COPD patients, being lowest in the patients with the highest severity of COPD. (Uncoupling protein-3 content is decreased in peripheral skeletal muscle of patients with COPD H. R. Gosker, P. Schrauwen, M. K. C. Hesselink, G. Schaart, G. J. van der Vusse, E. F. M. Wouters, A. M. W. J. Schols European Respiratory Journal July 2003, 22 (1) 88-93; DOI: 10.1183/09031936.03.00089802)

In skeletal muscle cell lines, hypoxia induces a decrease in activity along with a drop in electron transport chain complex IV mRNA level and activity. In mice, chronic hypoxia has been shown to decrease resistance to fatigue and complex I-mediated mitochondrial respiratory rate. Accordingly, in healthy humans, this was associated with a reduction in muscle mass as well as a decrease in mitochondrial density and a drop in electron transport chain complex I and IV protein content along with a lower PGC-1α protein content. Moreover, decreased UCP3 protein content and heightened oxidative stress have also been reported in humans. The role of hypoxia in skeletal muscle mitochondrial changes during COPD is more difficult to assess per se. failed to find decreased muscle mass and endurance in hypoxic compared with non-hypoxic COPD patients. However, a correlation between arterial partial pressure of oxygen (inline image) and quadriceps endurance time was observed, showing that hypoxia is implicated in a dose-dependent manner in muscle fatigability during COPD. (Meyer, A., Zoll, J., Charles, A. L., Charloux, A., de Blay, F., Diemunsch, P., Sibilia, J., Piquard, F. and Geny, B. (2013), Skeletal muscle mitochondrial dysfunction during chronic obstructive pulmonary disease: central actor and therapeutic target. Experimental Physiology, 98: 1063-1078. https://doi.org/10.1113/expphysio1.2012.069468).

COPD is also associated with muscle atrophy. The atrophy of skeletal muscles with dysfunction in the aging process, referred to as sarcopenia, is a general phenomenon and seems to originate from a decline in neuronal control or malnutrition. The muscle dysfunction results not only in inactivity but also probably a decrease in energy expenditure and/or impaired cold tolerance, because skeletal muscle, the biggest organ in the body, plays crucial roles in energy metabolism and thermogenesis. The dissipation of the mitochondrial proton gradient in skeletal muscles has been reported to reach 50% of the resting metabolic rate. The capacity for thermogenesis also tends to be attenuated with age. The muscle dysfunction with aging contributes to increased adiposity and decreased cold tolerance. (Kontani Y, Wang Z, Furuyama T, Sato Y, Mori N, Yamashita H (2002) Effects of aging and denervation on the expression of uncoupling proteins in slow- and fast-twitch muscles of rats. J. Biochem. 132, 309-315.).

### Aging

Aging is usually defined as a time-dependent decline of maximal functionality that affects tissues and organs of the whole body and leads to an increased susceptibility to disease and risk of death. Post-mitotic tissues such as skeletal muscle are preferentially prone to the adverse effects of advancing age. It has also been pointed out that mitochondria are a key factor in this process, since they have a major role in energetic homeostasis so determining ATP availability in the cells. Indeed, dysfunctional mitochondria will be unable to meet cellular ATP demands, thus compromising the cellular ability to adapt to physiological stress imposed to skeletal muscle across the entire lifespan.) Crescenzo, R., Bianco, F., Mazzoli, A. et al. Alterations in proton leak, oxidative status and uncoupling protein 3 content in skeletal muscle subsarcolemmal and intermyofibrillar mitochondria in old rats. BMC Geriatr 14, 79 (2014). https://doi.org/10.1186/1471-2318-14-79) Studies show that UCP3 and the protein it encodes plays a significant role in the reduction of mitochondrial damage due to oxidative stress, thereby promoting proper mitochondrial function and improved maximal functionality.

### Methods for Treating Genetic Diseases Associated with Decreased UCP3 Activity

Some embodiments disclosed herein provide methods for treating genetic diseases associated with decreased UCP3 activity in a human subject in need thereof, comprising (optionally) identifying a human subject having a genetic disease and in need of an increased expression level of a UCP3 gene; and administering to the human subject an effective amount of a nitroxide antioxidant. In some embodiments, the methods disclosed herein are be used to treat a human subject that shows no symptoms of the genetic disease, but is at risk of having the genetic disease. Exemplary risk factors for genetic diseases include, but are not limited to, age, family history, health conditions, medical history, habits, or a combination thereof. In some embodiments, risk factors for genetic disease comprise a decreased expression level of UCP3.

In some embodiments, administering to the human subject an effective amount of the nitroxide antioxidant results in an increased expression level of a gene, for example UCP3. The gene associated with uncoupling protein 3 can be UCP3, UCP1, UCP2, UCP4, UCP5, or a homologue thereof. The treatment of the human subject with the effective amount of the nitroxide antioxidant results in an increased expression level of the gene. For example, the treatment results in an increased expression level of UCP3. The increased expression level of UCP3, increases the quantity of the encoded protein and improve mitochondrial function inhibited by decreased expression levels. The improved and corrected uncoupling activity and mitochondrial function reduces, prevents, or eliminates the signs and symptoms of a genetic disease associated with decreased UCP3 function, including the curing of the genetic disease.

In some embodiments, the levels of UCP3 in the connective tissue, muscle tissue, nervous tissue, and/or epithelial tissue may change after the nitroxide antioxidant is administered. Non-limiting examples of the connective tissue include dense connective tissue, loose connective tissue, reticular connective tissue, adipose tissue, cartilage, bone, and extracellular matrix. Non-limiting examples of the muscle tissue includes smooth muscle tissue, cardiac muscle tissue, and skeletal muscle tissue. Non-limiting examples of the nervous tissue include neural tissue of the central nervous system, neural tissue of the peripheral nervous system, the brain, spinal cord, cranial nerves, spinal nerves, and motor neurons. Non-limiting examples of the epithelial tissue include squamous epithelium, cuboidal epithelium, columnar epithelium, glandular epithelium, ciliated epithelium, and skin.

Non-limiting examples of genetic diseases associated with decreased UCP3 activity include Osteogenesis imperfecta, Spondyloepiphyseal dysplasia, Spondyloepimetaphyseal dysplasia, Achondrogenesis, hypochondrogenesis, Kniest dysplasia, Stickler syndrome, Ehlers-Danlos syndrome, Familial porencephaly, Hereditary angiopathy with nephropathy, aneurysms and muscle cramps syndrome, Benign familial haematuria, Alport syndrome, Leiomyomatosis, Bethlem myopathy, Ullrich congenital muscular dystrophy, Dystrophic epidermolysis bullosa, Corneal endothelial dystrophies Multiple epiphyseal dysplasia, Autosomal recessive Stickler syndrome, Schmid metaphyseal chondrodysplasia, Marshall syndrome, Otospondylomegaepiphyseal dysplasia Deafness, Junctional epidermolysis bullosa-other Knobloch syndrome

### Methods for Counteracting Treating a Disease Related to Aging

Some embodiments disclosed herein provide methods for counteracting age-related increase in gene expression or treating an age-related disease, comprising (optionally) identifying a human subject over the age of 35 and having a decreased expression level of UCP3 or an age-related disease; and administering to the human subject an effective amount of a nitroxide antioxidant. In some embodiments, the methods comprise determining the expression level of UCP3. The identification step and/or the determination step may not be necessary in some instances, such as where a decreased expression level of UCP3 is inferred from the human subject's age, family history, health conditions, medical history, habits, or a combination thereof. In some embodiments, the methods disclosed herein are used to treat a human subject shows no symptoms of an age-related disease, but is at risk of having an age-related disease. Exemplary risk factors for an age-related disease include, but are not limited to, age, family history, health conditions, medical history, habits, or a combination thereof. In some embodiments, risk factors for an age-related disease comprise a decreased expression level of UCP3.

In some embodiments, administering to the human subject an effective amount of the nitroxide antioxidant results in an increased expression level of a gene, for example UCP3. The gene associated with uncoupling protein 3 can be UCP3, UCP1, UCP2, UCP4, UCP5, or a homologue thereof. The treatment of the human subject with the effective amount of the nitroxide antioxidant results in a increased expression level of the gene. For example, the treatment results in an increased expression level of UCP3. The increased expression level of UCP3, corrects mitochondrial function and uncoupling activity to a healthy level within the cell. The corrected level of uncoupling activity and mitochondrial function results in a decrease in or disappearance of signs and symptoms of an age-related disease associated with decreased UCP3 function, including the curing of the age-related disease.

In some embodiments, the levels of UCP3 in the connective tissue, muscle tissue, nervous tissue, and/or epithelial tissue may change after the nitroxide antioxidant is administered. Non-limiting examples of the connective tissue include dense connective tissue, loose connective tissue, reticular connective tissue, adipose tissue, cartilage, bone, and extracellular matrix. Non-limiting examples of the muscle tissue includes smooth muscle tissue, cardiac muscle tissue, and skeletal muscle tissue. Non-limiting examples of the nervous tissue include neural tissue of the central nervous system, neural tissue of the peripheral nervous system, the brain, spinal cord, cranial nerves, spinal nerves, and motor neurons. Non-limiting examples of the epithelial tissue include squamous epithelium, cuboidal epithelium, columnar epithelium, glandular epithelium, ciliated epithelium, and skin.

Some embodiments disclosed herein provide methods for treating a disease related to aging in a human subject in need thereof, comprising (optionally) identifying a human subject over the age of 35 and having an age-related disease and having a decreased expression level of the UCP3 gene; and administering to the human subject an effective amount of a nitroxide antioxidant. Some embodiments disclosed herein provide methods for treating an individual having or at risk of developing a condition due to aging, comprising: identifying an individual over the age of 35; and administering to the individual an effective amount of a nitroxide antioxidant, whereby the expression level of the gene associated with uncoupling protein 3 is increased.

Non-limiting examples of age-related diseases include cancer, rheumatoid/osteoid arthritis, systemic lupus erythematosus (SLE), inflammatory bowel disease, Alzheimer' s disease, multiple sclerosis, atherosclerosis, cardiovascular disease, cataracts, dementia, osteoporosis, type 2 diabetes, hypertension.

### Methods for Increasing Expression Level of a Gene

Some embodiments disclosed herein provide methods for increasing the expression level of a gene in a human subject in need thereof, comprising (optionally) identifying a human subject having a decreased expression level of a UCP3 gene; and administering to the human subject an effective amount of a nitroxide antioxidant. Some embodiments disclosed herein provide methods for treating a disease associated with decreased UCP3 activity in a patient in need thereof, comprising (optionally) identifying a human subject having a decreased expression level of UCP3; and administering to the human subject an effective amount of a nitroxide antioxidant. The increased expression level may be age-related, or disease related. In some embodiments, the disease may be cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, hypertension, or any combination thereof. Some embodiments disclosed herein provide methods for treating an individual in need thereof, comprising (optionally) identifying a human subject over the age of 35 in need of an increased expression level of a UCP3 gene; and administering to the human subject an effective amount of a nitroxide antioxidant. In some embodiments, the methods comprise determining the expression level of UCP3. In some embodiments, the determination step comprises inferring decreased expression level of UCP3 based on the human subject's age, family history, health conditions, medical history, habits, or a combination thereof. In some embodiments, the methods disclosed herein are used to treat a human subject shows no symptoms of a disease associated with decreased UCP3 function, but is at risk of having a disease associated with decreased UCP3 function. Exemplary risk factors for a disease associated with decreased UCP3 function include, but are not limited to, age, family history, health conditions, medical history, habits, or a combination thereof.

In some embodiments, administering to the human subject an effective amount of the nitroxide antioxidant results in an increased expression level of a gene, for example a gene associated with uncoupling protein activity. The gene associated with uncoupling protein 3 can be UCP3. The treatment of the human subject with the effective amount of the nitroxide antioxidant results in an increased expression level of the gene. For example, the treatment can decrease the expression levels of UCP3. The increased expression of the gene counteracts the decrease in the expression level of the gene.

### Methods for Treating Cancer

Some embodiments disclosed herein provide methods for treating cancer in a human subject in need thereof, comprising (optionally) identifying a human subject having a cancer and in need of an increased expression level of a UCP3 gene; and administering to the human subject an effective amount of a nitroxide antioxidant. In some embodiments, the methods disclosed herein are used to treat a human subject that shows no symptoms of cancer, but is at risk of having cancer. Exemplary risk factors for cancer include, but are not limited to, age, family history, health conditions, medical history, habits, or a combination thereof. In some embodiments, risk factors for cancer comprise a decreased expression level of UCP3.

Non-limiting examples of the methods for identifying a human subject having a cancer include colonoscopy; sigmoidoscopy; and high-sensitivity fecal occult blood tests. In some embodiments, methods for identifying a human subject having a cancer include low-dose helical computed tomography; mammography; and pap test and human papillomavirus (HPV) testing. In some embodiments, methods for identifying a human subject having a cancer include alpha-fetoprotein blood test; breast magnetic resonance imaging (MRI); CA-125 test; clinical breast exams and regular breast self-exams; prostate-specific antigen (PSA) testing; skin exams; transvaginal ultrasound; and virtual colonoscopy. In some embodiments, methods for identifying a human subject having a cancer include barium enema; biopsy; bone marrow aspiration and biopsy; bone scan; breast MRI for early detection of breast cancer; breast MRI; colonoscopy; computed tomography (CT) scan; digital rectal exam (DRE); blood and platelets testing; bone marrow testing; umbilical cord blood testing; electrocardiogram (EKG) and echocardiogram; endoscopic techniques; fecal occult blood tests; magnetic resonance imaging (MRI); mammography; multi gated acquisition (MUGA) scan; papanicolaou (pap) test; positron emission tomography and computed tomography (PET-CT) scan; sigmoidoscopy; tumor marker tests; ultrasound; upper endoscopy. In some embodiments, methods for identifying a human subject having a cancer include DNA sequencing; detecting presence of single nucleotide polymorphism (SNIP); and detecting the presence of certain protein markers.

In some embodiments, administering to the human subject an effective amount of the nitroxide antioxidant results in an increased expression level of a gene, for example a gene associated with uncoupling protein activity. The gene associated with uncoupling protein 3 can be UCP3. The treatment of the human subject with the effective amount of the nitroxide antioxidant results in an increased expression of the gene. For example, the treatment results in an increased expression level of UCP3. The increased expression level of the gene can modulate mitochondrial function and uncoupling activity to a healthy rate and function. The improved mitochondrial function and uncoupling activity results in a decrease in or disappearance of signs and symptoms of the cancer, including the curing of the cancer.

Non-limiting examples of cancer include bladder and other urothelial cancers; breast cancer; cervical cancer; colorectal cancer; endometrial cancer; endometrial cancer; esophageal cancer; liver (hepatocellular) cancer; lung cancer; neuroblastoma cancer; oral cavity and oropharyngeal cancer; ovarian, fallopian tube, and primary peritoneal cancer; prostate cancer; skin cancer; stomach (gastric) cancer; and testicular cancer.

Non-limiting examples of cancer include acute lymphoblastic leukemia, adult; acute myeloid leukemia, adult; adrenocortical carcinoma; aids-related lymphoma; anal cancer; bile duct cancer; bladder cancer; brain tumors, adult; breast cancer; breast cancer and pregnancy; breast cancer, male; carcinoid tumors, gastrointestinal; carcinoma of unknown primary; cervical cancer; chronic lymphocytic leukemia; chronic myelogenous leukemia; chronic myeloproliferative neoplasms; cns lymphoma, primary; colon cancer; endometrial cancer; esophageal cancer; extragonadal germ cell tumors; fallopian tube cancer; gallbladder cancer; gastric cancer; gastrointestinal carcinoid tumors; gastrointestinal stromal tumors; germ cell tumors, extragonadal; germ cell tumors, ovarian; gestational trophoblastic disease; hairy cell leukemia; hepatocellular (liver) cancer, adult primary; histiocytosis, langerhans cell; hodgkin lymphoma, adult; hypopharyngeal cancer; intraocular (eye) melanoma; islet cell tumors, pancreatic neuroendocrine tumors; kaposi sarcoma; kidney (renal cell) cancer; kidney (renal pelvis and ureter, transitional cell) cancer; langerhans cell histiocytosis; laryngeal cancer; leukemia, adult acute lymphoblastic; leukemia, adult acute myeloid; leukemia, chronic lymphocytic; leukemia, chronic myelogenous; leukemia, hairy cell; lip and oral cavity cancer; liver cancer, adult primary; lung cancer, non-small cell; lung cancer, small cell; lymphoma, adult Hodgkin; lymphoma, adult non-hodgkin; lymphoma, aids-related; lymphoma, primary cns; malignant mesothelioma; melanoma; melanoma, intraocular (eye); merkel cell carcinoma; metastatic squamous neck cancer with occult primary; multiple myeloma and other plasma cell neoplasms; mycosis fungoides and the sézary syndrome; myelodysplastic syndromes; myelodysplastic/myeloproliferative neoplasms; myeloproliferative neoplasms, chronic; paranasal sinus and nasal cavity cancer; nasopharyngeal cancer; neck cancer with occult primary, metastatic squamous; non-hodgkin lymphoma, adult; non-small cell lung cancer; oral cavity cancer, lip oropharyngeal cancer; ovarian epithelial cancer; ovarian germ cell tumors; ovarian low malignant potential tumors; pancreatic cancer; pancreatic neuroendocrine tumors (islet cell tumors); pheochromocytoma and paraganglioma; paranasal sinus and nasal cavity cancer; parathyroid cancer; penile cancer; pheochromocytoma and paraganglioma; pituitary tumors; plasma cell neoplasms, multiple myeloma and other; breast cancer and pregnancy; primary peritoneal cancer; prostate cancer; rectal cancer; renal cell cancer; transitional cell renal pelvis and ureter; salivary gland cancer; sarcoma, Kaposi; sarcoma, soft tissue, adult; sarcoma, uterine; mycosis fungoides and the sézary syndrome; skin cancer, melanoma; skin cancer, nonmelanoma; small cell lung cancer; small intestine cancer; stomach (gastric) cancer; testicular cancer; thymoma and thymic carcinoma; thyroid cancer; transitional cell cancer of the renal pelvis and ureter; trophoblastic disease, gestational; carcinoma of unknown primary; urethral cancer; uterine cancer, endometrial; uterine sarcoma; vaginal cancer; and vulvar cancer.

In some embodiments, non-limiting examples of cancer include, but are not limited to, hematologic and solid tumor types such as acoustic neuroma, acute leukemia, acute lymphoblastic leukemia, acute myelogenous leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer (including estrogen-receptor positive breast cancer), bronchogenic carcinoma, Burkitt's lymphoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer (including small cell lung cancer and non-small cell lung cancer), lymphangioendothelio-sarcoma, lymphangiosarcoma, lymphoblastic leukemia, lymphoma (lymphoma, including diffuse large B-cell lymphoma, follicular lymphoma, Hodgkin's lymphoma and non-Hodgkin's lymphoma), malignancies and hyPerproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer (including hormone-insensitive (refractory) prostate cancer), rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), stomach cancer, squamous cell carcinoma, synovioma, sweat gland carcinoma, testicular cancer (including germ cell testicular cancer), thyroid cancer, Waldenstrom's macroglobulinemia, testicular tumors, uterine cancer, Wilms' tumor and the like.

Non-limiting examples of the cancer include acute lymphoblastic leukemia, childhood; acute myeloid leukemia/other myeloid malignancies, childhood; adrenocortical carcinoma, childhood; astrocytomas, childhood; atypical teratoid/rhabdoid tumor, childhood central nervous system; basal cell carcinoma, childhood; bladder cancer, childhood; bone, malignant fibrous histiocytoma of and osteosarcoma; brain and spinal cord tumors overview, childhood; brain stem glioma, childhood; (brain tumor), childhood astrocytomas; (brain tumor), childhood central nervous system atypical teratoid/rhabdoid tumor; (brain tumor), childhood central nervous system embryonal tumors; (brain tumor), childhood central nervous system germ cell tumors; (brain tumor), childhood craniopharyngioma; (brain tumor), childhood ependymoma; breast cancer, childhood; bronchial tumors, childhood; carcinoid tumors, childhood; carcinoma of unknown primary, childhood; cardiac (heart) tumors, childhood; central nervous system atypical teratoid/rhabdoid tumor, childhood; central nervous system embryonal tumors, childhood; central nervous system germ cell tumors, childhood; cervical cancer, childhood; chordoma, childhood; colorectal cancer, childhood; craniopharyngioma, childhood; effects, treatment for childhood cancer, late; embryonal tumors, central nervous system, childhood; ependymoma, childhood; esophageal tumors, childhood; esthesioneuroblastoma, childhood; ewing sarcoma; extracranial germ cell tumors, childhood; gastric (stomach) cancer, childhood; gastrointestinal stromal tumors, childhood; germ cell tumors, childhood central nervous system; germ cell tumors, childhood extracranial; glioma, childhood brain stem; head and neck cancer, childhood; heart tumors, childhood; hematopoietic cell transplantation, childhood; histiocytoma of bone, malignant fibrous and osteosarcoma; histiocytosis, langerhans cell; hodgkin lymphoma, childhood; kidney tumors of childhood, wilms tumor and other; langerhans cell histiocytosis; laryngeal cancer, childhood; late effects of treatment for childhood cancer; leukemia, childhood acute lymphoblastic; leukemia, childhood acute myeloid/other childhood myeloid malignancies; liver cancer, childhood; lung cancer, childhood; lymphoma, childhood Hodgkin; lymphoma, childhood non-Hodgkin; malignant fibrous histiocytoma of bone and osteosarcoma; melanoma, childhood; mesothelioma, childhood; midline tract carcinoma, childhood; multiple endocrine neoplasia, childhood; myeloid leukemia, childhood acute/other childhood myeloid malignancies; nasopharyngeal cancer, childhood; neuroblastoma, childhood; non-hodgkin lymphoma, childhood; oral cancer, childhood; osteosarcoma and malignant fibrous histiocytoma of bone; ovarian cancer, childhood; pancreatic cancer, childhood; papillomatosis, childhood; paraganglioma, childhood; pediatric supportive care; pheochromocytoma, childhood; pleuropulmonary blastoma, childhood; retinoblastoma; rhabdomyosarcoma, childhood; salivary gland cancer, childhood; sarcoma, childhood soft tissue; (sarcoma), ewing sarcoma; (sarcoma), osteosarcoma and malignant fibrous histiocytoma of bone; (sarcoma), childhood rhabdomyosarcoma; (sarcoma) childhood vascular tumors; skin cancer, childhood; spinal cord tumors overview, childhood brain and; squamous cell carcinoma (skin cancer), childhood; stomach (gastric) cancer, childhood; supportive care, pediatric; testicular cancer, childhood; thymoma and thymic carcinoma, childhood; thyroid tumors, childhood; transplantation, childhood hematopoietic; childhood carcinoma of unknown primary; unusual cancers of childhood; vaginal cancer, childhood; vascular tumors, childhood; and wilms tumor and other childhood kidney tumors.

Non-limiting examples of cancer include embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous system, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer.

### Methods for Treating Autoimmune Diseases

Some embodiments disclosed herein provide methods for treating an autoimmune disease in a human subject in need thereof, comprising (optionally) identifying a human subject having an autoimmune disease and in need of an increased expression level of a UCP3 gene; and administering to the human subject an effective amount of a nitroxide antioxidant. In some embodiments, the methods disclosed herein are used to treat a human subject shows no symptoms of an autoimmune disease, but is at risk of having an autoimmune disease. Exemplary risk factors for an autoimmune disease include, but are not limited to, age, family history, health conditions, medical history, habits, or a combination thereof. In some embodiments, risk factors for an autoimmune disease comprise a decreased expression level of UCP3.

In some embodiments, Autoimmunity is the system of immune responses of an organism against its own healthy cells and tissues. Any disease that results from such an aberrant immune response is termed an “autoimmune disease”. Prominent examples include celiac disease, diabetes mellitus type 1, sarcoidosis, systemic lupus erythematosus (SLE), Sjogren's syndrome, eosinophilic granulomatosis with polyangiitis, Hashimoto's thyroiditis, Graves' disease, idiopathic thrombocytopenic purpura, Addison's disease, rheumatoid arthritis (RA), ankylosing spondylitis, polymyositis (PM), and dermatomyositis (DM). Autoimmune diseases are very often treated with steroids

In some embodiments, administering to the human subject an effective amount of the nitroxide antioxidant results in an increased expression level of a gene, for example a gene associated with uncoupling protein activity. The gene associated with uncoupling protein 3 can be UCP3. The treatment of the human subject with the effective amount of the nitroxide antioxidant results in an increased expression level of the gene. For example, the treatment results in an increased expression level of UCP3. The increased expression levels of UCP3, increases mitochondrial function and uncoupling activity resulting in a decrease in or disappearance of signs and symptoms of the autoimmune disease, including the curing of the autoimmune disease. In some embodiments, the increased expression level of UCP3, improves mitochondrial function. The improved mitochondrial function results in a decrease in or disappearance of signs and symptoms of the autoimmune disease, including the curing of the autoimmune disease.

Non-limiting examples of autoimmune diseases include rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma). The human antibodies, and antibody portions of the present application can be used to treat humans suffering from autoimmune diseases, in particular those associated with inflammation, including, rheumatoid spondylitis, allergy, autoimmune diabetes, autoimmune uveitis.

Non-limiting examples of autoimmune diseases include acquired immunodeficiency disease syndrome (AIDS), autoimmune lymphoproliferative syndrome, hemolytic anemia, inflammatory diseases, and thrombocytopenia, acute or chronic immune disease associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer' s disease, amyotrophic lateral sclerosis, anemia, angina pectoris, ankylosing spondylitis associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycaemia, autoimmune neutropaenia, autoimmune thrombocytopaenia, autoimmune thyroid disease, B cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinaemia), conjunctivitis, connective tissue disease associated interstitial lung disease, contact dermatitis, Coombs positive haemolytic anaemia, cor pulmonale, Creutzfeldt-Jakob disease, cryptogenic autoimmune hepatitis, cryptogenic fibrosing alveolitis, culture negative sepsis, cystic fibrosis, cytokine therapy associated disorders, Crohn's disease, dementia pugilistica, demyelinating diseases, dengue hemorrhagic fever, dermatitis, dermatitis scleroderma, dermatologic conditions, dermatomyositis/polymyositis associated lung disease, diabetes, diabetic arteriosclerotic disease, diabetes mellitus, Diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine, receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram negative sepsis, gram positive sepsis, granulomas due to intracellular organisms, group B streptococci (GBS) infection, Grave's disease, haemosiderosis associated lung disease, hairy cell leukemia, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpurea, Hepatitis A, Hepatitis B, Hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopaenia, idiopathic pulmonary fibrosis, idiopathic thrombocytopaenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anaemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease associated lung disease, monoclonal gammopathy, multiple myeloma, multiple systems degenerations (Mencel Dejerine-Thomas Shi-Drager and Machado-Joseph), myalgic encephalitis/Royal Free Disease, myasthenia gravis, microscopic vasculitis of the kidneys, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, Non-alcoholic Steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, Progressive supranucleo Palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynaud's disease, Refsum's disease, regular narrow QRS tachycardia, Reiter's disease, renal disease NOS, renovascular hypertension, reperfusion injury, restrictive cardiomyopathy, rheumatoid arthritis associated interstitial lung disease, rheumatoid spondylitis, sarcoidosis, Schmidt's syndrome, scleroderma, senile chorea, Senile Dementia of Lewy body type, sepsis syndrome, septic shock, seronegative arthropathies, shock, sickle cell anemia, Sjogren's disease associated lung disease, Sjorgren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, spondyloarthopathy, sporadic, polyglandular deficiency type I sporadic, polyglandular deficiency type II, Still's disease, streptococcal myositis, stroke, structural lesions of the cerebellum, Subacute sclerosing panencephalitis, sympathetic ophthalmia, Syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, systemic sclerosis, systemic sclerosis-associated interstitial lung disease, T-cell or FAB ALL, Takayasu's disease/arteritis, Telangiectasia, Th2 Type and Th1 Type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy and the like.

### Nitroxide Antioxidant

Nitroxide antioxidants describes a group of stable organic molecules, containing the nitroxyl group >N—O. with an unpaired electron. They have a low molecular weight, are non-toxic, do not elicit immunogenic effects on cells and easily diffuse through cell membranes. Their biological activity as antioxidants is related to the regulation of redox state in the cells. Nitroxides can undergo cyclic oxidation or reduction reactions. Their antioxidant activity is related to several mechanisms such as the direct scavenging of free radicals, transition metal ion oxidation. In addition, nitroxides exhibit superoxide dismutase (SOD)-like activity, modulate its catalase-like activity and ferroxidase-like activity, and are the inhibitors of free radical reactions such as lipid peroxidation. Nitroxides have dynamic beneficial impact on all cellular processes from inhibition of oxidative stress and reducing inflammation, while under certain conditions they may also lead to its intensification, for example, in tumor cells. The different beneficial impact on cellular processes provides each cell with necessary support to prevent or reverse diseases and conditions through optimizing cellular activity and associated biological processes in a healthy state and promoting cell death in diseases such as cancer.

Cyclic nitroxides, also known as aminoxyls or nitroxyls, are stable free radicals stabilized by methyl groups at the a position in five-membered pyrrolidine, pyrroline or oxazolidine and six-membered piperidine ring structures. The methyl groups confer stability to the nitroxide radicals by preventing radical-radical dismutation and also limit access to reactive substances, which can quench the radical species. The substituent groups on the ring (denoted by R—) produce a diverse range of compounds that can be directed to specific hydrophilic or hydrophobic regions in the cellular microenvironment. The redox transformations between the oxidation states of nitroxide, hydroxylamine and the oxoammonium cation acts as an efficient redox couple, which can support catalytic processes via reversible electron redox reactions. (Soule, Benjamin P et al. “The chemistry and biology of nitroxide compounds.” Free radical biology & medicine vol. 42,11 (2007): 1632-50. doi:10.1016/j.freeradbiomed.2007.02.030).

The mechanism of action exerted by nitroxide antioxidants is very unique. In particular, nitroxide antioxidant function is characterized by a catalytic mechanism of action associated with a single-electron redox cycle. Their reduction results in the generation of hydroxylamine and oxidation in oxoammonium ion; meanwhile both reactions are reversible and repetitive such that the ratio of free radicals suppressed by nitroxide antioxidants is significantly higher than natural antioxidant processes within a cell. Hydroxylamine also exhibits antioxidant properties because it is easily oxidized to nitroxide. As mentioned above, the nitroxides devoid of electrical charge easily diffuse through the cell membranes, thus they can also inactivate the reactive oxygen species formed in the cells and modulate the concentration of intracellular nitric oxide. Their molecular structure and composition make nitroxide antioxidants additionally efficacious in tissues that prevent transport of different molecules, such as neuronal tissue across the blood brain barrier.

Non-limiting examples of the nitroxide antioxidant include 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Amin omethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, 2,2,6,6-tetramethyl-4-oxo-1-piperidinyloxy (TEMPONE), 1-Hydroxy-2,2,6,6-tetramethyl-4-oxo-piperidine. HCl (TEMPONE-H), 1,2-dipalmitoyl-sn-glycero-3-phospho(tempo)choline (TEMPO PC), (4-[N,N- dimethyl-N-(2-hydroxyethyl)]ammonium-2,2,6,6-tetramethylpiperidine-1-oxyl (TEMPO Choline), and the like.

The use of other nitroxide compounds is also contemplated. Nitroxide stable radicals demonstrate effective antioxidative activity in various biological systems ranging from molecular, cellular, and laboratory animal level. Nitroxides have been reported to catalyze O2. dismutation through two different catalytic pathways including reductive and oxidative reaction mechanisms. Conversely, kinetics analysis of rapid mixing stopped flow experiments de-signed to measure the effect of nitroxides on superoxide decay did not reveal any SOD activity, leading to the conclusion that nitroxides act as free radical scavengers.

Studies have shown that unlike other antioxidants, nitroxides are characterized by a catalytic mechanism of action associated with a single-electron redox cycle. Their reduction results in the generation of hydroxylamine and oxidation in oxoammonium ion; meanwhile both reactions are reversible. Hydroxylamine also exhibits antioxidant properties because it is easily oxidized to nitroxide. Nitroxide antioxidants undergo redox cycles. They are easily reduced to hydroxylamines and oxidized to oxoammonium salts.

According to certain embodiments the nitroxide compound can be selected from the following formulas:

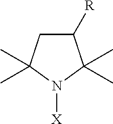

wherein X is selected from O— and OH, and R is selected from COOH, CONH, CN, and CH2NH2;

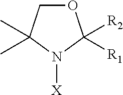

wherein X is selected from O— and OH, and R1 is selected from CH3 and spirocyclohexyl, and R2 is selected from C2H5 and spirocyclohexyl;

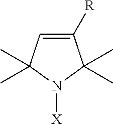

wherein X is selected from O— and OH and R is selected from CONH; and

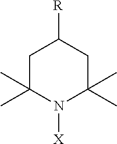

wherein X is selected from O— and OH and R is selected from H, OH, and NH2.

Suitable nitroxide compounds can also be found in Proctor, U.S. Pat. No. 5,352,442, and Mitchell et al., U.S. Pat. No. 5,462,946, both of which are hereby incorporated by reference in their entireties.

In some embodiments, the nitroxide antioxidant has a general formula:

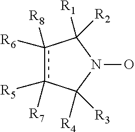

wherein the dashed line denotes a saturated bond or an unsaturated bond, wherein when the dashed line denotes an unsaturated bond, R7 and R8 are absent; R1-R4 are each independently a C1-4-alkyl, or alternatively, R1 and R2, and/or R3 and R4, together form a 3-7-membered alicyclic ring; and R5-R8 are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino,

In some embodiments, the nitroxide antioxidant includes or is associated with (e.g., binds to or is conjugated with) a bioeffector molecule. For example, the bioeffector molecule is a targeting subunit bound to the nitroxide antioxidant, such as a mitochondrial targeting subunit. A targeting subunit can direct activity of the nitroxide antioxidant to a predetermined location within or on the cell. Non-limiting examples of mitochondrial targeting bioeffector molecules includes triphenylphosphine (TPP), gramicidin, and any functional group effectively charged to be attracted to the polarized mitochondria.

In some embodiments, the nitroxide antioxidant is structurally cyclic having a ring structure including a nitroxide molecule incorporated therein. In some embodiments, the nitroxide antioxidant is characterized as the nitroxide molecule functioning as the catalytic center.

### Dosage

In some embodiments, the nitroxide antioxidant, non-toxic salts thereof, acid addition salts thereof or hydrates thereof may be administered systemically or locally, usually by oral or parenteral administration. The doses to be administered can be determined depending upon, for example, age, body weight, symptom, the desired therapeutic effect, the route of administration, and the duration of the treatment. In the human adult, the dose per person at a time can be generally from about 0.01 to about 4000 mg, by oral administration, up to several times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time can be generally from about 0.01 to about 300 mg/kg via parenteral administration (preferably intravenous administration), from one to four or more times per day. Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration can also contemplated for from 1 to 24 hours per day to achieve a target concentration from about 0.01 mg/L to about 100 mg/L. Non-limiting examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does can depend upon various conditions, and there may be cases wherein doses lower than or greater than the ranges specified above are used.

### Compositions

The nitroxide antioxidant can be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. In such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, the nitroxide antioxidant is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Injections for parenteral administration include solutions, suspensions, emulsions and solids which are dissolved or suspended. For injections, the nitroxide antioxidant can be dissolved, suspended and emulsified in a solvent. The solvents include, for example, distilled water for injection, physiological salt solution, vegetable oil, propylene glycol, polyethylene glycol, alcohol such as ethanol, or a mixture thereof. Moreover the injections can also include stabilizing agents, agents to assist dissolution (e.g. glutamic acid, aspartic acid or POLYSORBATE80™), suspending agents, emulsifying agents, soothing agents, buffer agents, preserving agents, etc. They can be sterilized in the final process or manufactured and prepared by sterile procedure. They can also be manufactured in the form of sterile solid compositions, such as a freeze-dried composition, and they may be sterilized or dissolved immediately before use in sterile distilled water for injection or some other solvent.

Other compositions for parenteral administration include liquids for external use, and ointment, endermic liniments, inhale, spray, suppositories for rectal administration and pessaries for vaginal administration which comprise the nixtroxide antioxidant and are administered by methods known in the art.

Spray compositions can comprise additional substances other than diluents: e.g. stabilizing agents (e.g. sodium sulfite hydride), isotonic buffers (e.g. sodium chloride, sodium citrate or citric acid). A small aerosol particle size useful for effective distribution of the medicament can be obtained by employing self-propelling compositions containing the drugs in micronized form dispersed in a propellant composition. Effective dispersion of the finely divided drug particles can be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size can be less than about 5 microns. The propellant composition may employ, as the suspending agent, a fatty alcohol such as oleyl alcohol. The minimum quantity of suspending agent can be approximately 0.1 to 0.2 percent by weight of the total composition. The amount of suspending agent can be less than about 4 percent by weight of the total composition to maintain an upper particle size limit of less than 10 microns or 5 microns. Propellants that may be employed include hydrofluoroalkane propellants and chlorofluorocarbon propellants. Dry powder inhalation may also be employed.

### EXAMPLES

Some aspects of the embodiments discussed above are disclosed in further detail in the following examples, which are not in any way intended to limit the scope of the present disclosure.

In order to facilitate understanding, the specific embodiments are provided to help interpret the technical proposal, that is, these embodiments are only for illustrative purposes, but not in any way to limit the scope of the invention. Unless otherwise specified, embodiments do not indicate the specific conditions, are in accordance with the conventional conditions or the manufacturer's recommended conditions.

### Example 1. Effects of Tempol on Expression of Genes Associated with Uncoupling Protein Activity

To assess the effects of Tempol on gene expression, Tempol was administered to experimental mice at a dose of 5 mg/g of food from 14 months to 31 months after birth. Mice receiving the same food without the addition of Tempol were used as a negative control. At the age of 31 months, the experimental animals were sacrificed and the hearts were surgically removed. The expression of a broad spectrum of genes in the cardiac tissue was assessed using chip-based microarray technology. Such chips are well known in the art and are widely used to assess gene expression. The experimental results showed that UCP3 exhibited statistically significant increase in expression. This result is shown in Table 1.

TABLE 1
Genes Associated With UCP3 Exhibiting Decreased Expression
In Adipose Tissue After Tempol Administration

| Symbol | Gene title | Fold change | P-value |
|---|---|---|---|
| UCP3 | Uncoupling Protein 3 | 1.57 | 0.00 |

### Example 2. Treating Age-Related Increase in Gene Expression

A 70-kilogram human subject over the age of 65 is identified as having, or known to have, or suspected of having a decreased expression level of UCP3. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

### Example 3. Treating a Human Subject with Increased Gene Expression

A 70-kilogram human subject is identified as having, or known to have, or suspected of having a decreased expression level of UCP3. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

### Example 4. Treating a Human Subject with an Age-Related Disease

A 70-kilogram human subject over the age of 65 and having a cardiovascular disease is identified for a decreased expression level of UCP3. Or a 70-kilogram human subject over the age of 65 is known to have a cardiovascular disease and/or decreased expression level of UCP3. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

### Example 5. Treating a Human Subject at Risk of Developing Cancer

A 70-kilogram human subject at risk of developing colorectal cancer is identified for decreased expression level of UCP3. Or a 70-kilogram human subject is known to be at risk of developing colorectal cancer and/or have decreased expression level of UCP3. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

### Example 6. Treating a Human Subject at Risk of Developing an Autoimmune Disease

A 70-kilogram human subject at risk of developing an autoimmune disease (e.g., rheumatoid arthritis) is identified for decreased expression level of UCP3. Or a 70-kilogram human subject is known to be at risk of developing an autoimmune disease and/or have decreased expression level of UCP3. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

### Example 7. Treating a Human Subject at Risk of Developing a Condition Due to Aging

A 70-kilogram human subject of 45 years old at risk of developing a condition due to aging is identified. Or a 70-kilogram human subject of 45 years old is known to be at risk of developing a condition. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

### Example 8. Treating a Human Subject at Risk of Developing a Neruodegenerative Disease

A 70-kilogram human subject at risk of developing a neurodegenerative disease (e.g., Parkinson's Disease) is identified for decreased expression level of UCP3. Or a 70-kilogram human subject is known to be at risk of developing a neurodegenerative disease and/or have decreased expression level of UCP3. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

### Example 9. Treating a Human Subject Having an Infection

A 70-kilogram human subject having an infection (e.g., a bacterial, fungal, or viral infection) is identified for decreased expression level of UCP3. Or a 70-kilogram human subject is known to have an infection and/or have decreased expression level of UCP3. The human subject is administered a dose of 2000 mg of Tempol (or another nitroxide antioxidant) per day for 180 days. This may be administered in a single dose, or may be administered as a number of smaller doses over a 24-hour period: for example, four 500-mg doses at eight-hour intervals. Following treatment, the serum level of UCP3, is increased.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of increasing expression level of uncoupling protein 3 (“UCP3”) encoded by UPC3 gene, the method comprising:
identifying a human subject having a disease or condition associated with a decreased expression level of the UPC3, the disease or condition selected from the group consisting of obesity, heart disease, diabetes, muscle wasting disease, myocardial infarction, and reduced UCP3 activity atherosclerosis; and
administering an effective amount of a nitroxide antioxidant to the subject,
whereby the expression level of UCP3 is increased.

2. (canceled)

3. (canceled)

4. The method of claim 1, wherein the disease or condition is obesity.

5. The method of claim 1, wherein the disease or condition is heart disease.

6. The method of claim 1, wherein the disease or condition is diabetes.

7. The method of claim 1, wherein the disease or condition is muscle wasting disease.

8. (canceled)

9. The method of claim 1, wherein the disease or condition is myocardial infarction.

10. The method of claim 1, wherein the nitroxide antioxidant is 4-hydroxy-2,2,6,6- tetramethylpiperidine-1-oxyl (“TEMPOL”).

11. The method of claim 1, wherein the subject is over the age of 35.

12. The method of claim 1, wherein the subject is over the age of 55.

13. (canceled)

14. (canceled)

15. The method of claim 1, wherein the disease is aging.

16. The method of claim 1, wherein the disease or condition is reduced UCP3 activity atherosclerosis.

17. (canceled)

18. (canceled)

19. (canceled)

20. The method of claim 1, wherein the expression level of UCP3 is increased in cardiac tissue of the subject.

21. The method of claim 1, wherein the nitroxide antioxidant is selected from the group consisting of 2-ethyl-2,5,5-trimethyl-3-oxazolidine-1-oxyl (OXANO), 2,2,6,6-tetramethylpiperidine-1-oxyl (TEMPO), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, 2,2,6,6-tetramethyl-4-oxo-1-piperidinyloxy (TEMPONE), 1-Hydroxy-2,2,6,6-tetramethyl-4-oxo-piperidine HCl (TEMPONE-H), 1,2-dipalmitoyl-sn-glycero-3-phospho(tempo)choline (TEMPO PC), (4-[N,N-dimethyl-N-(2-hydroxyethyl)]ammonium-2,2,6,6-tetramethylpiperidine-1-oxyl (TEMPO Choline).

22. The method of claim 1, wherein the expression level of UCP3 is increased in cardiac tissue of the subject.
